(19) United States
(12) Patent Application Publication
(10) Pub. No.: US 2005/0222407 A1
(43) Pub. Date: Oct. 6, 2005

(54) Method for the preparation of ethylenically unsaturated compounds with lactam-blocked isocyanate groups

(76) Inventor: Jacobus Antonius Loontjens, Meerssen (NL)

Correspondence Address: MAYER, BROWN, ROWE & MAW LLP, 1909 K STREET, N.W., WASHINGTON, DC, 20006, US

(21) Appl. No.: 10/505,325

(22) PCT Filed: Feb. 20, 2003
(86) PCT No.: PCT/NL03/00134

§ 371 Date: Mar. 25, 2005

(30) Foreign Application Priority Data
Feb. 21, 2002 (NL) 1020029

(51) Int. Cl. C08F 8/30; C07D 223/10

(52) U.S. Cl. 540/531; 525/126

(57) ABSTRACT

The invention relates to a method for the preparation of ethylenically unsaturated lactam-blocked isocyanate compounds, by reacting an unsaturated amine-functional or hydroxy-functional compound with carbonylbislactam. These compounds can be polymerised and are subsequently cured by the built-in crosslinker to form thermosetting polymers which can be applied for instance in powder coatings.

The invention relates to a method for the preparation of ethylenically unsaturated compounds with one or more lactam-blocked isocyanate groups, which can be used for the preparation of (co)polymer compositions, which compositions can in turn be crosslinked to form network structures.

Isocyanates are commonly used as crosslinkers for polymers that contain hydroxy groups in the polymer backbone, resulting in the formation of a urethane network. They are very reactive relative to nucleophiles, such as for example alcohols, phenols and amines. This high reactivity does however sometimes cause problems. Isocyanates are for example unstable in a moist environment. When used as a crosslinking agent premature gelation in the extruder is the greatest problem. The reactivity can be reduced by protecting the isocyanate groups, hereinafter referred to as blocked isocyanate groups.

Blocked isocyanates have been known for some 50 years, but only during the last 20 years has the use thereof significantly increased. For a detailed literature survey of the applications of blocked isocyanates, see D. A. Wicks and Z. W. Wicks Jr, Blocked isocyanates III, Part B: Uses and applications of blocked isocyanates, Progress in Organic Coatings 41:1-83 (2001), Elsevier Science B.V. These compounds are prepared conventionally by preparing first a isocyanate from the reaction of phosgene and a amine, after which a blocking agent is added to protect the isocyanate group (—N═C═O). There is a large variety of blocking agents; many isocyanates can be blocked excellently with caprolactam (Wicks, as above, page 9 and 10). Caprolactam is cheap and furthermore has a low toxicity. Only at higher temperatures does deblocking of the isocyanate group occur; in the case of caprolactam at 175° C.

Ethylenically unsaturated compounds with blocked isocyanate groups are described in the literature by for instance T. Sadoun c.s., Makromol. Chem. 188:1367-1373 (1987). These authors describe the preparation of 2-isocyanatoethyl methacrylate, with the isocyanate group being blocked by phenol, propanone oxime, butanone oxime, benzophenone oxime or ε-caprolactam (referred to hereinafter as caprolactam, unless otherwise stated), in one step, respectively the preparation in two steps of 4-methyl-1,3-phenylene diisocyanate, with the first isocyanate group being blocked by phenol or caprolactam and the second group by 2-hydroxyethyl methacrylate.

Further, G. Clouet and T. Sadoun, in Pure Appl. Chem., A29:939-952 (1992), describe the (co)polymerisation of 2-isocyanatomethacrylate, with the isocyanate groups being protected by phenol, propanone oxime, benzophenone oxime and caprolactam. These authors also indicate that by addition of bifunctional compounds, such as diamines or dioles, three-dimensional systems can be formed.

A great disadvantage of the preparation of ethylenically unsaturated blocked isocyanate compounds according to the prior art is that until now it has always been necessary to start from the corresponding unprotected isocyanate compounds, which are generally very toxic and expensive. The toxicity of compounds such as 2-cyanatoethylmethacrylate [30674-80-7], H₂C═C(CH₃)C(O)OCH₂CH₂N═C═O, has been documented in detail. For these reasons ethylenically unsaturated blocked isocyanate compounds have not been used until now for the preparation and curing of polymers of the category of hydroxy-functional poly(meth)acrylates or hydroxy-functional polyesters. Instead, these polymers were prepared in separate steps and cured by addition of bifunctional compounds such as HDI (hexamethylene diisocyanate) or IPDI (isophorone diisocyanate) or trimers hereof blocked with caprolactam. Adding a separate crosslinker does not only mean an extra step in the preparation process, but also has the objection that the crosslinker cannot always be mixed homogeneously with the relevant polymer resin.

The object of the invention is to provide a new method in which this disadvantage is avoided and ethylenically unsaturated lactam-blocked isocyanates are prepared in an environmentally friendly and efficient way.

According to another object of the invention polymers are provided of the poly(meth)acrylate type with a ‘built-in’ crosslinker, so that no extra mixing stage is necessary in the extruder and furthermore a possible mixing problem is avoided.

Surprisingly it was found that these objectives can be achieved by preparing ethylenically unsaturated compounds with one or more lactam-blocked isocyanate groups, without starting from an isocyanate compound, with an amine- or hydroxy-containing compound that contains at least one second functional group being reacted with a carbonylbislactam compound and the obtained blocked isocyanate compound, containing said at least one second functional group, if this is a group other than a vinyl group, being further converted into an ethylenically unsaturated blocked isocyanate compound.

The invention therefore relates to a method for the preparation of an ethylenically unsaturated blocked isocyanate compound with the general formula (I): 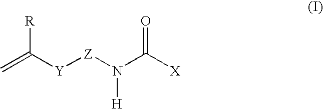
in which
R is hydrogen or methyl
X is a lactam group with formula (II): 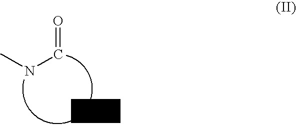
   in which
n is a whole number from 3 to 15,
Y is:
   carbonyl,
   phenyloxy (preferably 4-phenyloxy),
   (CH₂)ₘ, in which m is a whole number from 1 to 15 and the alkylene group can be substituted by one or more C₁₋₆ alkyl groups,
   carbonyloxy(CH₂)ₘ, in which m is a whole number from 1 to 15 and the alkylene group can be substituted by one or more C₁₋₆ alkyl groups,
   carbonyloxy(CH₂)ₘO(CH₂)ₚ, in which m and p are each separately a whole number from 1 to 15 and the respective alkylene group can be substituted by one or more C₁₋₆ alkyl groups,
   (CH₂)_{q}carbonylaza, in which q is a whole number from 0 to 15 and the alkylene group can be substituted by one or more C₁₋₆ alkyl groups, and
Z is a continuous bond or a carbonyl-(CH₂)ₙ group, in which n has the above-mentioned meaning, characterised in that
a) an amine-functional compound with formula (III): 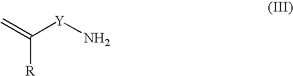
   in which R and Y have the above-mentioned meaning, or
b) a hydroxy-functional compound with formula (IV): 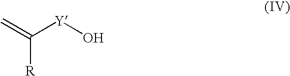
   in which R has the above-mentioned meaning and Y′ the same meaning as Y, except carbonyl, is reacted with a carbonylbislactam compound with formula (V): 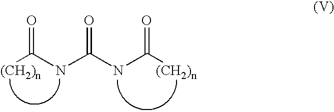
   in which n has the above-mentioned meaning.

Preferably carbonylbiscaprolactam (‘CBC’) is used as carbonylbislactam compound. This implies that in the above compounds with the formulas (I), (II) and (V) the value of n is preferably 5.

In the method defined above for the preparation of ethylenically unsaturated blocked isocyanates with formula (I) two main types can be distinguished: a) the reaction of an amine-functional compound with a carbonylbislactam compound with formula (V), with generally one of the lactam rings being split off, and b) the reaction of a hydroxy-functional compound with a carbonylbislactam compound with formula (V), with usually one of the lactam rings being opened. In the first case Z in formula (I) is a continuous bond, which means that in formula (I) Z represents a bond connecting Y and N directly to each other, and in the second case Z is a carbonyl alkylene group. In both cases the remaining part of the carbonylbislactam compound according to formula (V) constitutes the desired blocked isocyanate group in the compounds with formula (I), without use of an unblocked isocyanate compound. Generally the reaction of the amine-functional compounds with carbonylbislactam can be carried out without a catalyst, because the reactivity of the amine compounds to be used is usually adequate. If desired a suitable catalyst can still be added to promote the reaction further. For the reaction of the less reactive hydroxy-functional compounds a catalyst generally is necessary however. Suitable catalysts are for instance acids and bases, including Lewis acids and Lewis bases.

Examples of acids, including Lewis acids, that are suitable as a catalyst are LiX, Sb₂O₃, GeO₂ en As₂O₃, BX₃, MgX₂, BiX₃, SnX₄, SbX₅, FeX₃, GeX₄, GaX₃, HgX₂, ZnX₂, AlX₃, TiX₄, MnX₂, ZrX₄, R₄NX, R₄PX, HX, where X═I, Br, Cl, F, OR, acetylacetonate, or a compound according to formula (a) 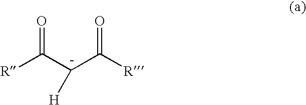
in which formula R″ and R′″ are independently chosen from the series comprising alkyl, aryl, alkoxy and aryloxy and R=alkyl or aryl. Brönstedt acids such as H₂SO₄, HNO₃, HX′ (where X′═I, Br, Cl, F), H₃PO₄, H₃PO₃, RH₂PO₂, RH₂PO₃, R[(CO)OH]ₙ, where n=1-6 are also suitable.

Examples of (Lewis) bases which are suitable as a catalyst are: alkali or earth alkali metal hydrides, hydroxides, C₁₋₂₀ alkoxides and phenolates, NR″″ₙH₄₋ₙOH (R″″═C₁₋₂₀ alkyl or aryl), triamines, such as triethylamine, tributylamine and trioctylamine, and cyclic amines, such as diazabicyclo[2,2,2]octane (DABCO), dimethylaminopyridine (DMAP), guanidine and morpholine.

A further aspect of the present invention, as an alternative to the method of the invention, relates to first reacting an amine-functional or a hydroxy-functional compound, which has furthermore at least one second functional group, with a carbonylbislactam compound according to formula (V), after which the resulting blocked isocyanate compound is further converted into an ethylenically unsaturated blocked isocyanate compound with formula (I). The second functional group is chosen from the group of hydroxyl, amine at a secondary carbon atom, secondary amine and an unsaturated group. A suitable amine-functional compound with a second functional group for example is hydroxyalkylamine, with the hydroxy group usually being terminal. This type of compound, the production of which is known or easily established for a person skilled in the art, reacts relatively quickly with carbonylbiscaprolactam (the amine group is more reactive than the hydroxy group, so that the desired link takes place in a predominant measure), after which the formed compound with the hydroxy functionality is converted into the desired compound with formula (I). An example of this preferred reaction is the reaction of a terminal hydroxyalkylamine with CBC, followed by the conversion of the obtained compound with (meth)acrylic acid or a reactive derivative thereof, for example the acid chloride, as is illustrated further in the examples 2a and 2b.

In a preferred method according to the present invention a compound with formula (I), in which X is a caprolactam group, Y a substituted or unsubstituted alkylene group and Z a continuous bond, is prepared by reacting the corresponding unsaturated alkylamine with CBC. An example of this reaction is the reaction of allylamine with CBC according to the following reaction equation:

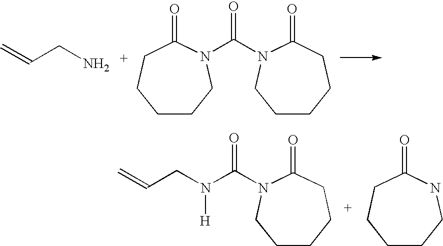

In another preferred method according to the present invention a compound with formula (I) is prepared, in which X is a caprolactam group, Y a carbonyl group and Z a continuous bond, by reacting the corresponding (meth)acrylamide with CBC. An example of this reaction is represented by the following reaction equation:

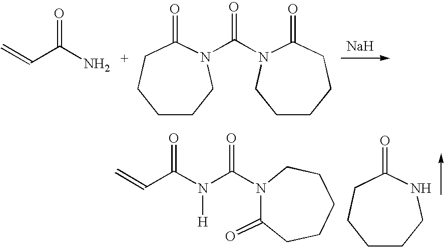

In yet another preferred method according to the present invention a compound with formula (I), in which X is a caprolactam group, Y a substituted or unsubstituted carbonyloxyalkyelene group and Z an oxycarbonyl(C₅)alkylene group, is prepared by reacting the corresponding hydroxyalkyl(meth)acrylate compound with CBC. An example of this reaction is represented by the following reaction equation:

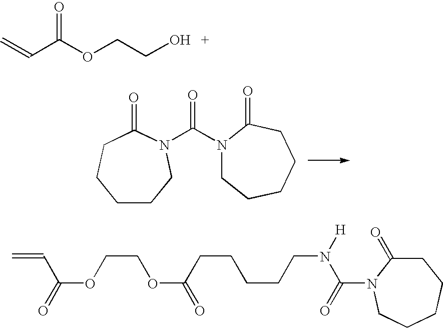

According to an alternative embodiment of the method for the preparation of a compound with formula (I) according to the present invention first an intermediate product is prepared by reacting an amine-functional or a hydroxy-functional compound, which furthermore comprises at least one second functional group but no unsaturated bond, with a carbonylbislactam compound according to formula (V), after which the obtained blocked isocyanate compound is further converted into an ethylenically unsaturated blocked isocyanate compound with formula (I).

Other compounds with formula (I), which have not been described in detail herein, can be prepared by a person skilled in the art without problems in a way known for the synthesis of analogous compounds, with the help of the present specifications and further with application of the general and specific knowledge of the person skilled in the art in this field.

The reaction conditions for the execution of the method according to the invention are not especially critical and can therefore be chosen by the person skilled in the art within fairly broad limits. Usually the reactions are carried out in a suitable solvent at a temperature which is chosen within broad limits and usually lies between room temperature and the boiling temperature of the solvent. A suitable solvent is for example toluene or xylene, with a reaction temperature which preferably lies in the range of 50-150° C., more preferably in the range of 70-150° C. The reaction time with the said solvent and the said reaction temperature is approximately 1 to 3 hours for the reaction of CBC with an amine-functional compound and approximately 3 to 5 hours for a compound with a hydroxy-functional compound.

The reactions can also be carried out without solvent in a melt of the reaction components, with the components being mixed, for example in a suitable reactor. In that case the reaction temperature preferably lies in the range of 100-150° C. and the reaction time is preferably 1 to 3 hours.

The preparation of the carbonylbislactam to be used, for example by reaction of the relevant lactam with phosgene, is described in the literature. See for example WO-A-98/47940. The preparation of N, N′-carbonylbiscaprolactam in benzene in the presence of a tertiary alkylamine as acid scavenger, is described for example in JP-A-42017832. Carbonylbiscaprolactam is commercially obtainable from DSM in Geleen, the Netherlands.

The preparation of the other starting materials described above is known from the literature or can be performed by a person skilled in the art in a way known for the preparation of analogous compounds.

The invention further relates to a thermosetting copolymer which contains more than one lactam-blocked isocyanate group and also at least two functional groups of the hydroxy and/or amine type per unit. These can be for example (co)polymers of the type of poly(meth)acrylates, which can be prepared in suitable way by (co)polymerisation of the above-defined ethylenically unsaturated compounds with one or more lactam-blocked isocyanate groups according to formula (I). For the copolymerisation compounds of the type of hydroxyalkyl(meth)acrylates, alkyl(meth)acrylates and/or styrene are very suitable. Due to the built-in lactam-blocked isocyanate groups in the compounds with formula (I) no separate crosslinker needs to be added for curing and the polymers can be cured without further additives whether or not immediately after the formation of the polymers by raising the temperature. A further advantage of the application of the lactam-blocked isocyanates according to the invention is that as crosslinkers these bring about no or only a small reduction of the glass transition temperature, which is important especially for the application in powder coatings.

Preferably the polymerisation takes place at a temperature in the range of 50 to 100° C. The reaction time of the polymerisation is also dependent on the type of initiator and is usually 2 to 10 hours. The curing is usually carried out at a temperature of 150-200° C. and the curing time usually is of the order of 10 to 30 minutes.

The polymers according to the invention can be applied for many kinds of purposes. Preferably they are applied in powder coatings. For the specific applications the usual additives such as pigments and flow agents can be added as desired during or after the preparation of the polymers.

The invention is now illustrated further by the following examples, which are intended exclusively as illustration of the invention and not to limit the invention in any way.

### EXAMPLE 1

Reaction of Allylamine with Carbonylbiscaprolactam

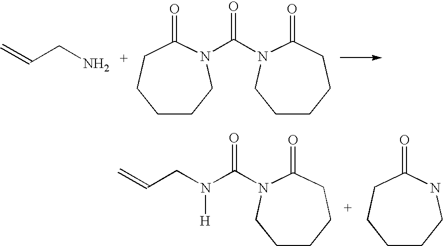

In a 250 ml flask, provided with a cooler, 5 g CBC (0.02 mole) was dissolved in 50 ml ethyl acetate (10% solution) at 50° C. A solution of 5.70 g allylamine (0.1 mole) in 50 ml ethyl acetate was added dropwise. With thin-layer chromatography (TLC; eluent ethylacetate:hexane 1:4; Rf_{CBC}=0.17 and Rf_{product}=0.32) it was established that after a night of reaction the CBC was completely converted. The solution was shaken out three times with a saturated aqueous NaCl solution to remove the excess allylamine and the formed caprolactam. The organic layer was dried with sodium sulphate. After removal of the sodium sulphate by filtration the solution was concentrated on a falling-film type evaporator. The yield of the desired product amounted to 2.5 g (65%).

¹H-NMR (CDCl₃) δ: 1.5-1.8 (b, 6H, —CH₂—), 2.6-2.8 (m, 2H, —CH₂—C═O), 3.7-4.0 (m, 4H, —CH₂—NH en —CH₂—N—C═O), 5.0-5.2 (m, 2H, C═CH₂), 5.7-5.9 (m, 1H, ═CH—CH₂), 9.1-9.5 (b, 1H, NH).

### EXAMPLE 2a

Reaction of Propanolamine with Carbonylbiscaprolactam, by Conversion with Acryloyl Chloride

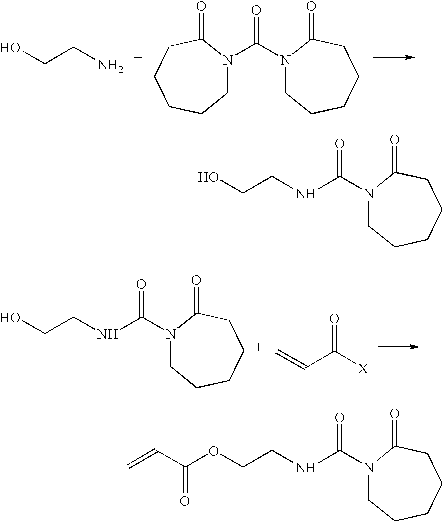

7.5 g (0.1 mole) propanolamine and 25.2 g (0.1 mole) carbonylbiscaprolactam were dissolved in 100 ml toluene. The solution was heated during 5 hours at 70° C. The mixture was cooled down to room temperature, after which 9.1 g (0.1 mole) acryloylchloride (X═Cl, in the above reaction) and 10.1 g (0.1 mole) triethylamine were added. The mixture was kept at 20° C. for 4 hours. The caprolactam and triethylamine.HCl salt, which had been released during the reaction, were removed by extracting the mixture twice with 50 ml water. The product was isolated by distilling toluene off at 90° C. and 25 mbar pressure.

### EXAMPLE 2b

Example 2a was repeated with acrylic acid instead of acryloyl chloride. Thus, 7.5 g (0.1 mole) propanolamine and 25.2 g (0.1 mole) carbonylbiscaprolactam were dissolved in 100 ml toluene. The solution was heated for 5 hours at 70° C. Hereafter 7.2 g (0.1 mole) acrylic acid, 0.25 g hydroquinone monomethylether and 0.25 g p-toluenesulfonic acid were metered in. The mixture was heated to 120° C. and the water was distilled off azeotropically for 4 hours. The mixture was cooled down to room temperature and extracted twice with 50 ml water. The monomer was isolated by distilling toluene off at 90° C. and 25 mbar pressure.

### EXAMPLE 3

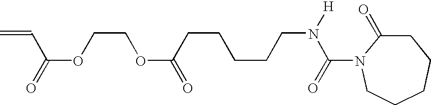

13 g (0.1 mole) hydroxyethylmethacrylate, 25.2 g (0.1 mole) carbonylbiscaprolactam, 0.25 g Zr(OBu)₄ and 0.025 g hydroquinone monomethylether were heated in a three-neck flask in a nitrogen atmosphere to 120° C. The reaction was ended after three hours and the mixture was analysed subsequently with IR and ¹H NMR. The mixture contained 81 wt % of the blocked isocyanate compound according to the structure as mentioned in the introduction of this example.

### EXAMPLE 4

Copolymerisation of Hydroxy Ethylacrvlate (HEA), Methylacrvlate (MA) and Caprolactam Blocked Isocyanopropylacrvlate (C-BIPA)

Polymerisations were carried out in a three-neck flask, provided with a stirrer and a nitrogen purge. Tetrahydrofuran (THF), which was used as solvent, had been dried previously on NaH. The monomers, as specified in Table 1, were polymerised in different ratios in 250 ml THF under nitrogen, with AIBN (azoisobutyronitrile) as initiator. For this polymerisation the ratio of the monomer relative to the initiator is 50:1.

The different compositions produced are shown in the the following Table 1.

TABLE 1
Composition of the copolymer

| | quantity | quantity | quantity | quantity | quantity | quantity | quantity | quantity |
|---|---|---|---|---|---|---|---|---|
| Monomer | (Mol %) | (g) | (Mol %) | (g) | (Mol %) | (g) | (Mol %) | (g) |
| MA | 95% | 16.34 g | 90% | 15.51 g | 85% | 14.63 g | 80% | 13.76 g |
| HEA | 2.5% | 0.60 g | 5% | 1.15 g | 7.5% | 1.73 g | 10% | 2.22 g |
| (C-BIPA) | 2.5% | 1.38 g | 5% | 2.66 g | 7.5% | 4.05 g | 10% | 5.36 g |

The polymers were isolated by evaporating the solvent THF at 50° C.

### EXAMPLE 5

Copolymerisation of Hydroxyethyl Methacrylate (HEMA), Methyl Methacrylate_(MMA) and Caprolactam Blocked Isocyanopropylacrylate (C-BIPA).

Polymerisations were carried out in a three-neck flask, provided with a stirrer and a nitrogen purge. Tetrahydrofuran (THF), which was used as solvent, was previously dried on NaH. The monomers, as specified in Table 2, were polymerised in 250 ml THF under nitrogen with AIBN as initiator. For these polymerisations the ratio of monomer relative to the initiator is 50:1. The different compositions produced are shown in the following Table 2.

TABLE 2
Composition of the copolymer

| | quantity | quantity | quantity | quantity | quantity | quantity | quantity | quantity |
|---|---|---|---|---|---|---|---|---|
| Monomer | (Mol %) | (g) | (Mol %) | (g) | (Mol %) | (g) | (Mol %) | (g) |
| MMA | 95% | 19.33 g | 90% | 18.12 g | 85% | 17.05 g | 80% | 16.04 g |
| HEMA | 2.5% | 0.65 g | 5% | 1.30 g | 7.5% | 2.00 g | 10% | 2.66 g |
| (C-BIPA) | 2.5% | 1.35 g | 5% | 2.72 g | 7.5% | 4.15 g | 10% | 5.43 g |

### EXAMPLE 6

Gel Experiments

Gel experiments are indicative of the crosslink behaviour of the coating resins. Gel experiments were carried out with approximately one gram of copolymer. The catalyst, dibutyltin dilaurate (see table for mass %), was added as a solution in THF to the polymer, after which the solvent was removed by heating. The polymer was heated to 180° C. and the gel point was determined as the moment when the product behaves no longer as a melt, but becomes a lump. The results are presented in Table 3.

TABLE 3
Results of gel experiments

| Incorporation | | Required cross- |
|---|---|---|
| specification | Amount of catalyst | linking time |
| (mol % HEA) | (mass %) | (seconds) |
| 2.5 | 1 | 330 |
| 2.5 | 0.5 | 250 |
| 2.5 | 0.1 | 280 |
| 2.5 | 0 | 510 |
| 5.0 | 1 | 185 |
| 5.0 | 0.5 | 150 |
| 5.0 | 0.1 | 225 |
| 7.5 | 1 | 105 |
| 7.5 | 0.5 | 110 |
| 7.5 | 0.1 | 100 |
| 10 | 1 | 195 |
| 10 | 0.5 | 204 |
| 10 | 0.1 | 252 |

The table suggests that the gel times are of the order of 2-10 minutes. This is in agreement with what is expected of a good crosslinking system.

1. Method for the preparation of an ethylenically unsaturated blocked isocyanate compound with the general formula (I): 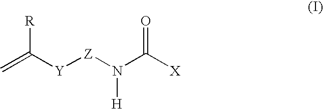
in which
R is hydrogen or methyl
X is a lactam group with formula (II): 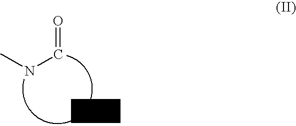
in which n is a whole number from 3 to 15,
Y is:
carbonyl,
phenyloxy (preferably 4-phenyloxy),
(CH₂)ₘ in which m is a whole number from 1 to 15 and the alkylene group can be substituted by one or more C₁₋₆ alkyl groups,
carbonyloxy(CH₂)ₘ, in which m is a whole number from 1 to 15 and the alkylene group can be substituted by one or more C₁₋₆ alkyl groups,
carbonyloxy(CH₂)ₘO(CH₂)ₚ, in which m and p are each separately a whole number from 1 to 15 and the respective alkylene group can be substituted by one or more C₁₋₆alkyl groups,
(CH₂)_{q}carbonylaza, in which Q is a whole number from 0 to 15 and the alkylene group can be substituted by one or more C₁₋₆ alkyl groups, and
Z is a continuous bond or a carbonyl-(CH₂)ₙ group, in which n has the above-mentioned meaning,
wherein
a) an amine-functional compound with formula (m): 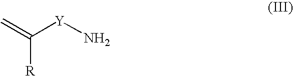
in which R and Y have the above-mentioned meaning, or
b) a hydroxy-functional compound with formula (IV): 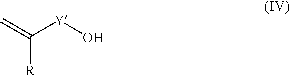
in which R has the above-mentioned meaning and Y′ the same meaning as Y, except carbonyl,
is reacted with a carbonylbislactam compound with formula (V): 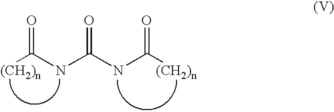
in which n has the above-mentioned meaning.

2. Method according to claim 1, wherein carbonylbiscaprolactam is used as carbonylbislactam compound.

3. Method for the preparation of an ethylenically unsaturated blocked isocyanate compound with the general formula (I): 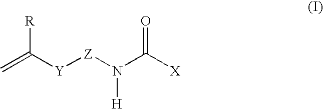
in which R, X, Y and Z have the meaning defined in claim 1, wherein an amine-functional or a hydroxy-functional compound, which further comprises at least one second functional group chosen from the group of hydroxyl, amine at a secondary carbon atom and secondary amine but no unsaturated bond, is reacted with a carbonylbislactam compound according to formula (V), 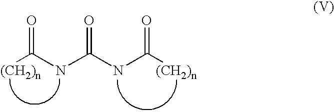
after which the obtained blocked isocyanate compound is further converted into an ethylenically unsaturated blocked isocyanate compound with formula (I).

4. Method according to claim 1, wherein a compound with formula (I) is prepared, in which X is a caprolactam group, Y a substituted or unsubstituted alkylene group and Z said continuous bond, by reacting the corresponding unsaturated alkylamine with carbonylbiscaprolactam.

5. Method according to claim 1, wherein a compound with formula (I) is prepared, in which X is a caprolactam group, Y a carbonyl group and Z said continuous bond, by reacting the corresponding (meth)acrylamide with carbonylbiscaprolactam.

6. Method according to claim 1, wherein a compound with formula (I) is prepared, in which X is a caprolactam group, Y a substituted or unsubtituted carbonyloxyalkyelene group and Z an oxycarbonyl(C₅)alkylene group, by reacting the corresponding hydroxyalkyl(meth)acrylate compound with carbonylbiscaprolactam.

7. Method according to claim 3, wherein a compound with formula (I) is prepared, in which X is a caprolactam group, Y a substituted or unsubtituted carbonyloxyalkyelene group and Z said continuous bond, by reacting a substituted or unsubtituted alkylamine compound, which has at least one second functional group, chosen from the group of hydroxyl, amine at a secondary carbon atom, secondary amine and an unsaturated group, with carbonylbiscaprolactam and thereby converting the obtained lactam-blocked isocyanate compound into an ethylenically unsaturated lactam-blocked isocyanate.

8. Method according to claim 7, wherein the alkylamine compound with a second functional group is hydroxyalkylamine and the obtained lactam-blocked isocyanate compound is converted with (meth)acrylic acid into an ethylenically unsaturated lactam-blocked isocyanate compound.
